(19) United States
(12) Patent Application Publication
(10) Pub. No.: US 2006/0123500 A1
(43) Pub. Date: Jun. 8, 2006

(54) Methods of prescreening cells for nuclear transfer procedures

(75) Inventors: Yann Echelard, Jamaica Plain, MA (US); Li-How Chen, Acton, MA (US)

Correspondence Address: GTC BIOTHERAPEUTICS, INC., 175 CROSSING BOULEVARD, SUITE 410, FRAMINGHAM, MA, 01702, US

(73) Assignee: GTC Biotherapeutics, Inc.

(21) Appl. No.: 11/081,945

(22) Filed: Mar. 16, 2005

(30) Foreign Application Priority Data
Dec. 7, 2004 (WO) PCT/US04/40816

(51) Int. Cl. A01K 67/027; C12N 5/06; C12N 15/87

(52) U.S. Cl. 800/14; 800/18; 435/455; 435/353; 435/354

(57) ABSTRACT

The present invention provides for the production of transgenic animals through pre-screening methods designed to improve the efficiency of nuclear transfer and consequentially the production characteristics of transgenic mammals relative to proteins of interest. The invention is thus useful in the production of transgenic ungulate animals capable of producing desired biopharmaceuticals in their milk at higher yield than a comparable heterzygote or producing animals with better physiological attributes.

### PRIORITY CLAIM

This application claims the benefit of priority of PCT Application No. PCT/US04/40816, filed Dec. 7, 2004, the contents of which are incorporated herein by reference.

### FIELD OF THE INVENTION

The present invention relates to the development of methods that increase the efficiency of cell-line screening for use in the production of transgenic animals. In particular, the current invention provides a method for improving cell lines through pre-selection methods such that downstream nuclear transfer procedures are improved.

### BACKGROUND OF THE INVENTION

The present invention relates generally to the field of somatic cell nuclear transfer (SCNT) and to the creation of desirable transgenic animals. More particularly, it concerns improved methods for selecting, generating, and propagating superior somatic cell-derived cell lines, and using these transfected cells and cell lines to generate transgenic non-human mammalian animal species, especially for the production of ungulates. Typically these transgenic animals will be used for the production of molecules of interest, including biopharmaceuticals, antibodies and recombinant proteins that are the subject of the transgene(s) of interest.

Animals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, and are often too time-consuming, costly and unreliable to develop. Moreover, these processes are completely incapable of allowing a specific animal line from producing gene products, such as desirable protein therapeutics that are otherwise entirely absent from the genetic complement of the species in question (i.e., human or humanized plasma protein or other molecules in bovine milk).

The development of technology capable of generating transgenic animals provides a means for exceptional precision in the production of animals that are engineered to carry specific traits or are designed to express certain proteins or other molecular compounds of therapeutic, scientific or commercial value. That is, transgenic animals are animals that carry the gene(s) of interest that has been deliberately introduced into existing somatic cells and/or germline cells at an early stage of development. As the animals develop and grow the protein product or specific developmental change engineered into the animal becomes apparent, and is present in their genetic complement and that of their offspring.

One of the challenges created by the biotechnology revolution is the development of methods for the economical production of highly purified proteins at large scale. The expression of recombinant proteins in the milk of transgenic dairy animals appears particularly well suited for the economical production of complex polypeptides (Reviewed in Clark, 1998; Meade et al., 1998). To that end, transgenic sheep, cows, goats and even pigs have been generated.

To express a recombinant protein in the milk of a transgenic animal, first the gene encoding the protein of interest is fused to milk specific regulatory elements to generate the transgene. These DNA constructs have traditionally been introduced in the germline of dairy animals by pronuclear microinjection of one-cell embryos (Hammer et al., 1985; Bondioli et al., 1991; Ebert et al., 1991; Wright et al., 1991). Microinjected embryos, at various stage of development depending on the species, were then transferred to a surrogate mother. Up to (and often less than) 5-10% of offspring resulting from pronuclear microinjections in large animals carried the transgene. Following integration into the germline, the mammary gland-specific transgene, if expressed, becomes a dominant genetic characteristic that will be predictably inherited by offspring of the founder animal depending on its degree of mosaicism.

However, the introduction of transgenes in the germline of large animals has often proven challenging and very labor intensive. While successful and widely used, the pronuclear microinjection approach has had limited efficiency. Transgene integration into the genome of founder animals is low and the frequent generation of mosaics (Wilkie et al., 1986; Burdon and Wall, 1992; Whitelaw et al., 1993) has sometimes complicated the expansion of transgenic herds (Williams et al., 1998; 2000). Transgenic founders often carry multiple integration sites, frequently with various degrees of mosaicism. Moreover, in the cases where the co-integration of multiple transgenes is necessary, for example for the expression of recombinant antibodies (Pollock et al., 1999), generation of animals carrying only one of the transgenes, or only one of the transgene within a specific chromosomal integration site, further decreases the frequency of “useful” founders.

The discovery that cultured cell lines can efficiently function as karyoplast donors for nuclear transfer has expanded the range of possibilities for germline modification in large animals. First sheep (Campbell et al., 1996; Wilmut et al., 1997), then cattle (Cibelli et al., 1998), goats (Baguisi et al., 1999; Keefer et al., 2001), and pigs (Onishi et al., 2000; Polejaeva et al., 2000; Betthauser et al., 2000) have successively been generated by this technique. Nuclear transfer with transfected somatic cells allows a more controlled introduction of transgenes and, in some circumstances, can reduce the number of animals (egg donors and recipients) used during the foundering process. It also overcomes the problem of founder mosaicism. The ability to pre-select transgenic cell lines before the generation of cloned transgenic embryos by analyzing transgene integration sites is also valuable. It is particularly important for the transgenic production of recombinant monoclonal antibodies in milk where often several transgenes have to be expressed in the same secretory cells of the mammary epithelium at equivalent levels. Co-integration of the transgenes in the same chromosomal locus, to avoid segregation of heavy chain and light chain genes during herd propagation, is also desirable.

Prior art methods of nuclear transfer and microinjection have typically used embryonic and somatic cells and cell lines selected without regard to any objective factors tying cell quality relative to the procedures necessary for transgenic animal production.

Thus although transgenic animals have been produced by various methods in several different species, methods to readily and reproducibly produce transgenic animals capable of expressing a desired protein or biopharmaceutical in high quantity or demonstrating the genetic alteration or enhancement caused by the insertion of the transgene(s) at reasonable costs are still lacking.

Accordingly, a need exists for improved methods of transgenic animal generation. The methods of the invention are typically applied to primary somatic cells, in the context of nuclear transfer, for the accelerated generation of a herd of transgenic animals useful in the production of recombinant proteins in milk.

### SUMMARY OF THE INVENTION

Briefly stated, the current invention provides a method for the accelerated production of transgenic animals. The method involves prescreening cells destined for transgenic procedures for abnormalities. By eliminating problem cell lines the resulting transgenic animal technologies are improved in efficiency. Thereafter, the methods of the invention include transfecting a selected non-human mammalian cell-line with a given transgene construct containing at least one DNA encoding a desired gene; selecting a cell line(s) in which the desired gene has been inserted into the genome of that cell or cell-line; and, performing a nuclear transfer procedure to generate a transgenic animal heterzygous for the desired gene.

An additional step that may performed according to the invention is to expand the biopsied cell-line obtained from the heterozygous animal in cell and/or cell-line in culture. An additional step that may performed according to the invention is to biopsy the heterozygous transgenic animal.

Alternatively a nuclear transfer procedure can be conducted to generate a mass of transgenic cells useful for research, serial cloning, or in vitro use. In a preferred embodiment of the current invention surviving cells are characterized by one of several known molecular biology methods including without limitation FISH, Southern Blot, PCR. The methods provided above will allow for the accelerated production of herd homozygous for desired transgene(s) and thereby the more efficient production of a desired biopharmaceutical.

Alternatively, the current invention allows for the production of genetically desirable livestock or non-human mammals.

In an alternate embodiment of the current invention multiple proteins can be integrated into the genome of a transgenic cell line that has been pre-screened to remove the possibility of cell line abnormalities, and/or screened after selected procedures to remove cell lines that become abnormal after the integration of a genetic sequence of interest. Successive rounds of transfection with another the DNA for an additional gene/molecule of interest (e.g., molecules that could be so produced, without limitation, include antibodies, and desired biopharmaceuticals).

Additionally these molecules could utilize different promoters that would be actuated under different physiological conditions or would lead to production in different cell types. The beta casein promoter is one such promoter turned on during lactation in mammary epithelial cells, while other promoters could be turned on under different conditions in other cellular tissues.

In addition, the methods of the current invention will allow the accelerated development of one or more homozygous animals that carry a particularly beneficial or valuable gene, enabling herd scale-up and potentially increasing herd yield of a desired protein much more quickly than previous methods. Likewise the methods of the current invention will also provide for the replacement of specific transgenic animals lost through disease or their own mortality. It will also facilitate and accelerate the production of transgenic animals constructed with a variety of DNA constructs so as to optimize the production and lower the cost of a desirable biopharmaceutical. In another objective of the current invention homozygous transgenic animals are more quickly developed for xenotransplantation purposes or developed with humanized Ig loci.

### BRIEF DESCRIPTION OF THE DRAWINGS

FIG. 1 Shows a flowchart of the methods involved in practicing the invention.

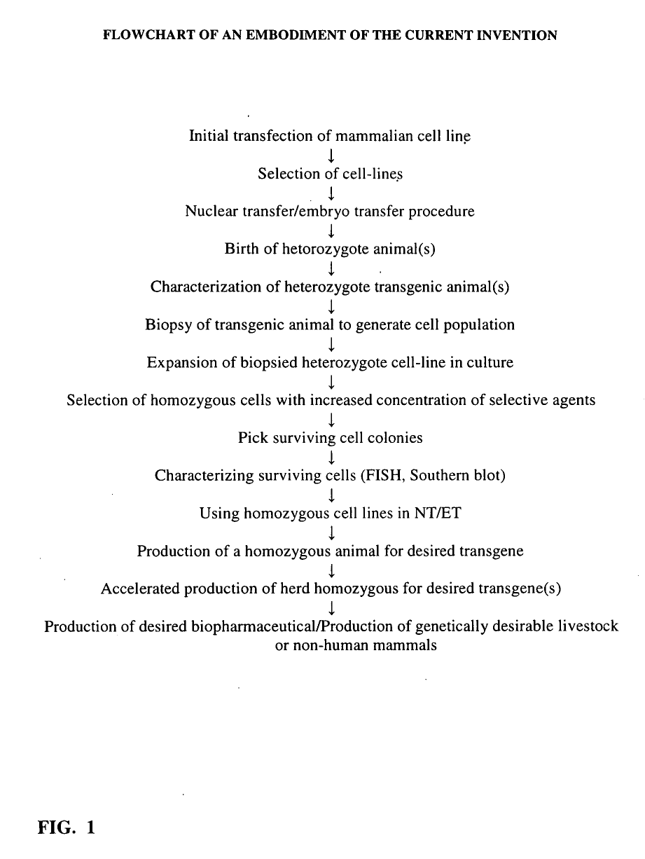

FIG. 2 Shows A Generalized Diagram of the Process of Creating Cloned Animals through Nuclear Transfer.

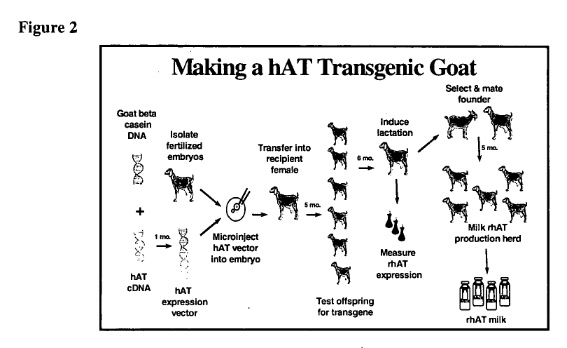

### DETAILED DESCRIPTION

The following abbreviations have designated meanings in the specification:

Abbreviation Key:

| | |
|---|---|
| Somatic Cell Nuclear Transfer | (SCNT) |
| Cultured Inner Cell Mass Cells | (CICM) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |

Explanation of Terms:
Bovine—Of or relating to various species of cows.
Caprine—Of or relating to various species of goats.
Cell Couplet—An enucleated oocyte and a somatic or fetal karyoplast prior to fusion and/or activation.
Cytocholasin-B—A metabolic product of certain fungi that selectively and reversibly blocks cytokinesis while not effecting karyokinesis.
Cytoplast—The cytoplasmic substance of eukaryotic cells.
Fusion Slide—A glass slide for parallel electrodes that are placed a fixed distance apart. Cell couplets are placed between the electrodes to receive an electrical current for fusion and activation.
Karyoplast—A cell nucleus, obtained from the cell by enucleation, surrounded by a narrow rim of cytoplasm and a plasma membrane.
Nuclear Transfer—or “nuclear transplantation” refers to a method of cloning wherein the nucleus from a donor cell is transplanted into an enucleated oocyte.
Ovine—of, relating to or resembling sheep.
Parthenogenic—The development of an embryo from an oocyte without the penetrance of sperm
Porcine—of, relating to or resembling swine or pigs
Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.
Selective Agent—Compounds, compositions, or molecules that can act as selection markers for cells in that they are capable of killing and/or preventing the growth of a living organism or cell not containing a suitable resistance gene. According to the current invention such agents include, without limitation, Neomycin, puromycin, zeocin, hygromycin, G418, gancyclovir and FIAU. Preferably, for the current invention increasing the dosage of the selective agent will kill all cell lines that only contain one integration site (e.g., heterozygous animals and/or cells).
Somatic Cell—Any cell of the body of an organism except the germ cells.
Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.
Transgenic Organism—An organism into which genetic material from another organism has been experimentally transferred, so that the host acquires the genetic information of the transferred genes in its chromosomes in addition to that already in its genetic complement.
Ungulate—of or relating to a hoofed typically herbivorous quadraped mammal, including, without limitation, sheep, swine, goats, cattle and horses.
Xenotransplantation—any procedure that involves the use of live cells, tissues, and organs from one animal source, transplanted or implanted into another animal species (typically humans) or used for clinical ex-vivo perfusion

According to the present invention, the accelerated development of superior transgenic genotypes of mammals with improved efficiencies, characteristics, or enhanced biopharmaceutical production, including caprines and bovines, are provided.

Prescreening of Somatic Cells Prior to Nuclear Transfer

This invention relates to the genetic characterization of transfected somatic cells prior to use as karyoplasts donors in nuclear transfer prior to engage in the nuclear transfer procedure. Analysis of several murine, caprine and bovine transgenic lines has shown that in the cases of transgenes incorporating the chicken globin (Chung et al., 1993) insulator sequence there is a correlation between the number of copies of trangenes at the integration sites and the expression level of the transgene. In both murine and caprine cases, it was observed that if the copy number of the integrated transgene is very high (>20 copies) this can lead to over expression of the transgene, affecting the health of the animal. In the case of mammary gland specific transgenes very high copy numbers can lead to over-production of the transgene-encoded protein in milk (>20 gIl), leading lactations that are reduced or inexistant. On the other hand low-copy number transgene integrations (1 to 2 copies), or transgene integrations located on the X chromosome will often lead to lower expression level of the transgene (<1 gIl), often incompatible with successful commercialization of these transgenic lines.

The ability to use transfected somatic cells for nuclear transfer opens the possibility to prescreen the cell line prior to their use in the generation of transgenic animals. This, combination of using insulator-containing transgenes to provide copy-number dependent expression and somatic cell nuclear transfer allows us to perform pre-screening of the cell lines in order to select donor cell lines that will a greatest chance of giving rise to transgenic animals with expression characteristics that are both compatible with the physiology of the animal (not too high) and with successful commercialization of the transgenic line (not too low).

Several methods can be employed for genotyping screening of the transgected somatic cells:
PCR assays
Southern blot analysis
Fluorescence in situ hybridization.
Giemsa staining (standard karyotyping)
BrdU incorporation for sister chromatid exchange analyses.

Of these methods, FISH is preferred for use with the current invention since qualitative information is obtained: number and chromosomal location of integration sites, percentage mosaicism of the donor cell line; as well as semi-quantitative information. By comparing the intensity of the signal given by the transgene integration sites with either endogenous signals or control lines, one can evaluate the copy number. Southern blotting helps evaluate in the transgene is rearranged. FISH can also be used to identify the specific chromosome onto which the transgene(s) is (are) integrated. There is an obvious value in screening out Y-chromosome specific integrations (only males are transgenic), and it is also a good idea to reject X-chromosome integration, since sometimes the transgene is associated with X inactivation. As stated earlier, prescreening is particularly useful in the generation of cell lines containing multiple transgenes (as for full antibody production). In this case it is particularly important to determine if all the transgenes are present in the cell lines, and if they are present in the same locus.

Pre-screening methods, according to the current invention, also allow the elimination of cell lines with chromosomal abnormalities. Such cell lines inevitably arise in culture, and if employed will give rise to non-viable or poorly viable animals. Examples of abnormalities that can be looked for are: chromosomal complement (using Giemsa staining), evidence of chromosome breakage and translocation (using Giemsa staining or several banding procedures); abnormal sex chromosome complements (X0, XXY, XYY etc. . . . ), evidence of chromosomal instability (sister chromatin exchange).

### EXPERIMENTAL SUMMARY

Cell lines are first transfected with the transgene of interest using standard procedures (Ex: electroporation, lipofection). Recombinant clones are then isolated using standard methods (for example drug resistance), giving rise to isolated colonies. An aliquot (a few thousand cells) for each colony is frozen, to stop cell division and prevent the onset of senescence. For each colony, the remaining cells are kept in culture, expanded and genotyped. Clonal cell lines that have low-copy integration (<2 copies) or very high-copy integration (>20 copies) are then identified and discarded; only cell lines that have preferred copy number (for example 3-20 copies) are retained and used in nuclear transfer procedures aiming at creating transgenic animals.

In the case where one was to decide to favor lower expression of the transgene, for examples if the transgene encodes a very bioactive protein for which even modest expression levels could lead to deleterious effects on the animal health, it would be possible to look for low-copy integration sites. Once a promising candidate is identified, the frozen aliquot can then be thawed, expanded and used in nuclear transfer procedures at will.

Another factor to keep in mind is that this characterization of the transgenic donor cell lines has to occur fairly rapidly. Donor cells are primary cells, they have to be used before as nuclear transfer karyoplasts before the growth arrest brought on by the onset of senescence (with goat fibroblasts, typically 30 to 50 cell divisions). According to the current invention methods have been developed that will allow the rapid identification of promising transfected candidates, freeze an aliquot, and pursue genotyping on the remainder of the cells. The ability to freeze early small aliquots of cells is important, since it maximizes the number of generations that we can use to perform somatic cell nuclear transfer. Possibly (although the data on this is probably not very strong) the use of a “younger” cell line could also lead to a healthier offspring.

Accordingly, the inventors have successfully applied the preferred methods of the current invention to the cloning of goats and are working on transitioning this technology to other species.

Following selection of recombinant colonies, cells are isolated and expanded, with aliquots frozen for long-term preservation according to procedures known in the field. The selected transgenic cell-lines can be characterized using standard molecular biology methods (PCR, Southern blotting, FISH). Cell lines carrying a transgene(s) of the appropriate copy number, generally with a single integration site (although the same technique could be used with multiple integration sites) can then be used as karyoplast donors in a somatic cell nuclear transfer protocol. Following nuclear transfer, and embryo transfer to a recipient animal, and gestation, live transgenic offspring are obtained. Typically this transgenic offspring carries only one transgene integration on a specific chromosome, the other homologous chromosome not carrying an integration in the same site. Hence the transgenic offspring is heterozygous for the transgene.

Following the increased selection, resistant colonies are genotyped (either by FISH or Southern blotting) to insure that the resulting cell line carries twice as many copies of the transgene and that both chromosome carry the integration. In addition karyotyping should be performed to insure that the cell line as the normal chromosomal complement.

### EXAMPLE 1

Protocol Using G418 Selection:
I. Plate primary cells at 2×10⁵/10 cm petri dish.
II. Set up 2 petris for every concentration of G418. Optimum concentrations of G418 will vary from cell line to cell line, example:
   1.2″
   1.5″
   2.0″
   2.5″
   3.0″
Add the drug at the same time you plate the cells. No need to let the cells settle down first.
III. Feed plates daily for the next five days with fresh medium+drug. After ˜5 days most of the cells will be dead, so feeding can be dropped back to every other day or so.
IV. Pick 6-24 of the best looking clones from the highest concentration of G418 onto 24-well wells.
V. Freeze and expand for DNA and karyotyping. Immobilize cells on filters for interphase FISH.

In another embodiment of the current invention, following the initial transfection, and isolation of the cell line, the cells be subjected immediately to increased selection to generate the homozygous cell line prior to generate an offspring.

Experiments:

In addition, the present invention relates to cloning procedures in which cell nuclei derived from somatic or differentiated fetal or adult mammalian cell lines are utilized. These cell lines include the use of serum starved differentiated fetal or adult caprine or bovine (as the case may be) cell populations and cell lines later re-introduced to serum as mentioned infra, these cells are transplanted into enucleated oocytes of the same species as the donor nuclei. The nuclei are reprogrammed to direct the development of cloned embryos, which can then be transferred to recipient females to produce fetuses and offspring, or used to produce cultured inner cell mass cells (CICM). The cloned embryos can also be combined with fertilized embryos to produce transfer. However, these methods do not generate Ca⁺² oscillations patterns similar to sperm in a typical in vivo fertilization pattern.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported. The stage of cell cycle that the karyoplast is in at time of reconstruction has also been documented as critical in different laboratories methodologies (Kasinathan et al., Biol. Reprod. 2001; Lai et al., 2001; Yong et al., 1998; and Kasinathan et al., Nature Biotech. 2001).

### MATERIALS AND METHODS

Estrus synchronization and superovulation of donor does used as oocyte donors, and micro-manipulation was performed as described in Gavin W. G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al., 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Enucleation and reconstruction was performed with, but may also be performed without, staining the oocytes with Hoechst 3342 at approximately 0.1-5.0 ug/ml and ultraviolet illumination of the genetic material/metaphase plate. Following enucleation and reconstruction, the karyoplast/cytoplast couplets were incubated in equilibrated Synthetic Oviductal Fluid medium supplemented with fetal bovine serum (1% to 15%) plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

Fusion was performed using a fusion slide constructed of two electrodes. The fusion slide was placed inside a fusion dish, and the dish was flooded with a sufficient amount of fusion buffer to cover the electrodes of the fusion slide. Cell couplets were removed from the culture incubator and washed through fusion buffer. Using a stereomicroscope, cell couplets were placed equidistant between the electrodes, with the karyoplast/cytoplast junction parallel to the electrodes. In these experiments an initial single simultaneous fusion and activation electrical pulse of approximately 2.0 to 3.0 kV/cm for 20 (can be 20-60) μsec was applied to the cell couplets using a BTX ECM 2001 Electrocell Manipulator. The fusion treated cell couplets were transferred to a drop of fresh fusion buffer. Fusion treated couplets were washed through equilibrated SOF/FBS, then transferred to equilibrated SOF/FBS with (1 to 10 μg/ml) or without cytochalasin-B. The cell couplets were incubated at 37-39° C. in a humidified gas chamber containing approximately 5% CO₂ in air.

Starting at approximately 30 minutes post-fusion, karyoplast/cytoplast fusion was determined. Fused couplets received an additional single electrical pulse (double pulse) of approximately 2.0 kV/cm for 20 (20-60) μsec starting at 1 hour (15 min-1 hour) following the initial fusion and activation treatment to facilitate additional activation. Alternatively, another group of fused cell couplets received three additional single electrical pulses (quad pulse) of approximately 2.0 kV/cm for 20 μsec, at fifteen-minute intervals, starting at 1 hour (15 min to 1 hour) following the initial fusion and activation treatment to facilitate additional activation. Non-fused cell couplets were re-fused with a single electrical pulse of approximately 2.6 to 3.2 kV/cm for 20 (20-60) μsec starting at 1 hours following the initial fusion and activation treatment to facilitate fusion. All fused and fusion treated cell couplets were returned to SOF/FBS with (1 to 10 μg/ml) or without cytochalasin-B. The cell couplets were incubated at least 30 minutes at 37-39° C. in a humidified gas chamber containing approximately 5% CO₂ in air.

Starting at 30 minutes following re-fusion, the success of karyoplast/cytoplast re-fusion was determined. Fusion treated cell couplets were washed with equilibrated SOF/FBS, then transferred to equilibrated SOF/FBS with (1 to 10 μg/ml) or without cycloheximide. The cell couplets were incubated at 37-39° C. in a humidified gas chamber containing approximately 5% CO₂ in air for up to 4 hours.

Following cycloheximide treatment, cell couplets were washed extensively with equilibrated SOF medium supplemented with bovine serum albumin (0.1% to 1.0%) plus 100 U/ml penicillin and 100 μg/ml streptomycin (SOF/BSA). Cell couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

The ability to pre-select a superior cell line to be used in a nuclear transfer program has remarkable implications. A significant amount of nuclear transfer work occurs with limited success as seen by the publications referenced in this document. In many of these publications a fair amount of work is done with very poor results or a complete lack of offspring born for individual cell (karyoplast) lines.

Paramount to the success of any nuclear transfer program is having adequate fusion of the karyoplast with the enucleated cytoplast. Equally important however is for that reconstructed embryo (karyoplast and cytoplast) to behave as a normal embryo and cleave and develop into a viable fetus and ultimately a live offspring. Results from this lab detailed above show that both fusion and cleavage either separately or in combination have the ability to predict in a statistically significant fashion which cell lines are favorable to nuclear transfer procedures. While alone each parameter can aid in pre-selecting which cell line to utilize, in combination the outcome for selection of a cell line is strengthened.

Goats.

The herds of pure- and mixed-breed scrapie-free Alpine, Saanen and Toggenburg dairy goats used as cell and cell line donors for this study were maintained under Good Agricultural Practice (GAP) guidelines.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/ml)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Transfected fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) for at least 6 hours prior to fusion to the enucleated oocytes.

Oocyte Collection.

Oocyte donor does were synchronized and superovulated as previously described (Gavin W. G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml).

Cytoplast Preparation and Enucleation.

All oocytes were treated with cytochalasin-B (Sigma, 5 μg/ml in SOF with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in SOF for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

Fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients.

All nuclear transfer embryos were cultured in 50 μl droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2μ (Lutalyse, Upjohn). Parturition occurred within 24 hours after treatment. Kids were removed from the dam immediately after birth, and received heat-treated colostrum within 1 hour after delivery.

Genotyping of Cloned Animals.

Shortly after birth, blood samples and ear skin biopsies are obtained from the cloned female animals (e.g., goats) and the surrogate dams for genomic DNA isolation. According to the current invention each sample may be first analyzed by PCR using primers for a specific transgenic target protein, and then subjected to Southern blot analysis using the cDNA for that specific target protein. For each sample, 5 μg of genomic DNA was digested with EcoRI (New England Biolabs, Beverly, Mass.), electrophoreses in 0.7% agarose gels (SeaKem®, Maine) and immobilized on nylon membranes (MagnaGraph, MSI, Westboro, Mass.) by capillary transfer following standard procedures known in the art. Membranes were probed with the 1.5 kb Xho I to Sal I hAT cDNA fragment labeled with ³²P dCTP using the Prime-It® kit (Stratagene, La Jolla, Calif.). Hybridization was executed at 65° C. overnight. The blot was washed with 0.2×SSC, 0.1% SDS and exposed to X-OMAT™ AR film for 48 hours.

The present invention allows for increased efficiency of transgenic procedures by increasing the number of potentially useful transgenic lines. Since it allows the rapid generation of transgenic animals with a substantial yield of recombinant protein production.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, hormones, plasma proteins, and other molecules of interest in the milk or other bodily fluid (i.e., urine or blood) of transgenic animals homozygous for a desired gene. Proteins capable of being produced in through the method of the invention include: antithrombin III, lactoferrin, urokinase, PF4, alpha-fetoprotein, alpha-1-antitrypsin, C-1 esterase inhibitor, decorin, interferon, ferritin, prolactin, CFTR, blood Factor X, blood Factor VIII, as well as monoclonal antibodies.

According to an embodiment of the current invention when multiple or successive rounds of transgenic selection are utilized to generate a cell or cell line homozygous for more than one trait such a cell or cell line can be treated with compositions to lengthen the number of passes a given cell line can withstand in in vitro culture. Telomerase would be among such compounds.

Accordingly, it is to be understood that the embodiments of the invention herein providing for an increased efficiency and speed in the production of transgenic animals are merely illustrative of the application of the principles of the invention. It will be evident from the foregoing description that changes in the form, methods of use, and applications of the elements of the disclosed method for the improved pre-selection of cell or cell lines for use in nuclear transfer or micro-injection procedures are novel and may be modified and/or resorted to without departing from the spirit of the invention, or the scope of the appended claims.

### LITERATURE CITED AND INCORPORATED BY REFERENCE

1. Alberio R, et al., (2001). Mammalian Oocyte Activation: Lessons from the Sperm and Implications for Nuclear Transfer, INT. J. DEV. BIOL. 2001; 45: 797-809.
2. Baguisi A, (1999) et al., Production of Goats by Somatic Cell Nuclear Transfer, NAT BIOTECH; 17: 456-461.
3. Behboodi, E., et al., Production Of Transgenic Goats By Adult Somatic Cell Nuclear Transfer, THERIOGENOLOGY 55, 254 (2001).
4. Betthauser, J., et al., Production of Cloned Pigs From In Vitro Systems, NATURE BIOTECH. 18, 1055-1059 (2000).
5. Bondioli, K. R., et al., Production of Transgenic Cattle by Pronuclear Injection, BIOTECHNOLOGY 16, 265-273 (1991).
6. Burdon, T. G., and Wall, R. J., Fate Of Microinjected Genes In Preimplantation Mouse Embryos. MOLEC. REPROD. DEVELOP. 33, 436-442 (1992).
7. Campbell, K. H. S., McWhir, J., Ritchie, W. A., Wilmut, I. (1996). Sheep cloned by nuclear transfer from a cultured cell line. Nature 380, 64-66.
8. Chen L. H., Behboodi, E., Reggio, B. C., Liem, H., Destrempes, M. M., Green, H. L., Ziomek, C. A., Denniston, R. A., Echelard, Y., Godke, R. A., Meade, H. M. (2001). Production of transgenic goats from a transfected cell line. Theriogenology 55, 259.
9. Chung, J. H., Whiteley, M., Felsenfeld G. (1993). A 5′ element of the chicken beta-globin domain serves as an insulator in human erythroid cells and protects against position effect in Drosophila. Cell 74, 505-514.
10. Cibelli, J. B., Stice, S. L., Golueke, P. J., Kane, J. J., Jerry, J., Blackwell, C., Ponce de Leon, F. A., Robl, J. M. (1998). Cloned transgenic calves produced from nonquiescent fetal fibroblasts. Science 280, 1256-1258.
11. Clark, A. J. (1998). The mammary gland as a bioreactor: Expression, processing, and production of recombinant proteins. I. Mammary Gland Biol. Neopl. 3, 337-350.
12. Ebert, K. M., Selgrath, J. P., DiTullio, P., Denman, J., Smith, T. E., Memon, M. A., Schindler, J. E., Monastersky, G. M., Vitale, J. A., Gordon, K. (1991). Transgenic production of a variant of human tissue-type plasminogen activator in goat milk: Generation of transgenic goats and analysis of expression. Bio/Technology 9, 835-838.
13. Echelard, Y. (1996). Recombinant protein production in the milk of transgenic animals. Curr. Opin. Biotech. 7, 536-540.
14. Emery, D. W., Yannaki, E., Tubb, J., Stamatoyannopoulos, G. (2000). A chromatin insulator protects retrovirus vectors from chromosomal position effects. Proc. Natl. Acad. Sci. U.S.A. 97, 9150-9155.
15. Fiering, S., Epner, E., Robinson, K., Zhuang, Y., Telling, A., Hu, M., Martin, D. I. K., Enver, T., Ley, T. J. & Groudine, M. (1995). Targeted deletion of S′1-1S2 of the murine beta-globin LCR reveals that it is not essential for proper regulation of the beta-globin locus. Genes Dev. 9, 2203-2213.
16. Gavin W. G., Pollock, D., Fell, P., Yelton, D., Cammuso, C., Harrington, M., Lewis Williams, J., Midura, P., Oliver, A., Smith, T. E., Wilburn, B., Echelard, Y., Meade H. (1997). Expression of the antibody hBR96 in the milk of transgenic mice and production of hBR96 transgenic goats. Theriogenology 47, 214.
17. Hammer, R. E., Pursel, V. G., Rexroad, C. E. Jr, Wall, R. J., Bolt, D. J., Ebert, K. M., Palmiter, R. D., Brinster, R. L. (1985). Production of transgenic rabbits, sheep and pigs by microinjection. Nature 315, 680-683.
18. Hug, B. A., Wessleschmidt, R. L., Fiering, S., Bender, M. A., Epner, E., Groudine, M., Ley, T. J. (1996). Analysis of mice containing a targeted deletion of beta-globin locus control region 5′ hypersensitive site 3. Molec. Cell. Biol. 16, 2906-2912.
19. Inoue, T., Yamaza, H., Sakai, Y., Mizuno, S., Ohno, M., Hamasaki, N., Fukumaki, Y. (1999). Position-independent human beta-globin gene expression mediated by a recombinant adeno-associated virus vector carrying the chicken beta-globin insulator. J. Hum. Genet. 44, 152-162.
20. Kasinathan P, (2001) et al., Effect of Fibroblast Donor Cell Age and Cell Cycle on Development of Bovine Nuclear Transfer Embryos In Vitro, BIOL REPROD.; 64(5): 1487-1493.
21. Kasinathan P, (2001) et al., Production of Calves from G1 Fibroblasts, NATURE BIOTECH; 19: 1176-1178.
22. Keefer, C. L., Baldassarre, H., Keyston, R., Wang, B., Bhatia, B., Bilodeau, A. S., Zhou, J. F., Leduc, M., Downey, B. R., Lazaris, A., Karatzas, C. N. (2001). Generation of Dwarf Goat (Ca pra hircus) Clones following nuclear transfer with transfected and nontransfected fetal fibroblasts and in vitro-matured oocytes. Biol. Reprod. 64, 849-856.
23. Kim, C. G., Epner, E. M., Forrester, W. C., Groudine, M. (1992). Inactivation of the human beta-globin gene by targeted insertion into the beta-globin locus control region. Genes Dev. 6, 928-938.
24. Maga, E. A., Murray, J. D. (1995). Mammary gland expression of transgenes and the potential for altering the properties of milk. Bio/Technology 13, 1452-1457.
25. McDevitt, M. A., Shivdasani, R. A., Fujiwara, Y., Yang, H., Orkin, S. H. (1997). A “knockdown” mutation created by cis-element gene targeting reveals the dependence of erythroid cell maturation on the level of transcription factor GATA-1. Proc. Natl. Acad. Sci. U. S. A. 94, 6781-6785.
26. Meade, H. M., Echelard, Y., Ziomek, C. A., Young, M. W., Harvey, M., Cole, E. S., Groet, S., Smith, T. E., Curling, J. M. (1998). Expression of recombinant proteins in the milk of transgenic animals. Gene Expression Systems: Using Nature For The Art of Expression, Fernandez, J. M., Hoeffler, J. P. eds, Academic Press, San Diego.
27. Newton, D. L., Pollock, D., DiTulio, P., Echelard, Y., Harvey, M., Wilburn, B., Williams, J., Hoogenboom, H. R., Raus, J. C. M., Meade, H. M., Rybak, S. M. (1999). Antitransferrin receptor antibody-RNase fusion protein expressed in the mammary gland of transgenic mice. I. Immunol. Methods 231, 159-167.
28. Olson, E. N., Arnold, H. H., Rigby, P. W. J., Wold, B. J. (1996). Know your neighbors: three phenotypes in null mutants of the myogenic bHLH gene MRF4. Cell 85, 1-4.
29. Onishi, A., Iwamoto, M., Akita, T., Mikawa, S., Takeda, K., Awata, T., Hanada, H., Perry, A. C. (2000). Pig cloning by microinjection of fetal fibroblast nuclei. Science. 289, 1188-1190.
30. Polejaeva, I. A., Chen, S. H., Vaught, T. D., Page, R. L., Mullins, J., Ball, S., Dai, Y., Boone, J., Walker, S., Ayares, D. L., Colman, A., Campbell, K. H. (2000). Cloned pigs produced by nuclear transfer from adult somatic cells. Nature 407, 86-90.
31. Pollock, D. P., Kutzko, J. P., Birck-Wilson, E., Williams, J. L., Echelard, Y., Meade H. M. (1999). Transgenic milk as a method for the production of recombinant antibodies. I. Immunol. Meth. 231, 147-157.
32. Potts, W., Tucker, D., Wood, H., Martin, C. (2000). Chicken beta-globin 5′HS4 insulators function to reduce variability in transgenic founder mice. Biochem. Biophys. Res. Comm. 273, 1015-1018.
33. Prioleau, M. N., Nony, P., Simpson, M., Felsenfeld, G. (1999). An insulator element and condensed chromatin region separate the chicken beta-globin locus from an independently regulated erythroid-specific folate receptor gene. EMBO J. 18, 4035-4048.
34. Reggio, B. C., James, A. N., Green, H. L., Gavin, W. G., Behboodi, E., Echelard, Y., Godke, R. A., Cloned Transgenic Offspring Resulting from Somatic Cell Nuclear Transfer in the Goat: Oocytes Derived from Both FSH-Stimulated and Nonstimulated Ovaries. Accepted for publication in Biol. Reprod.
35. Rivella, S., Callegari, I. A., May, C., Tan, C. W., Sadelain, M. (2000). The cHS4 insulator increases the probability of retroviral expression at random chromosomal integration sites. I. Virol. 74, 4679-4687.
36. Taboit-Dameron, F., Malassagne, B., Viglietta, C., Puissant, C., Leroux-Coyau, M., Chereau, C., Attal, J., Weill, B., Houdebine, L. M. (1999). Association of the 5′HS4 sequence of the chicken beta-globin locus control region with human EF1 alpha gene promoter induces ubiquitous and high expression of human CD55 and CD59 cDNAs in transgenic rabbits. Transgenic Res. 8, 223-235.
37. Walters, M. C., Fiering, S., Bouhassira, E. E., Scalzo, D., Goeke, S., Magis, W., Garrick, D., Whitelaw, E., Martin, D. I. (1999). The chicken beta-globin 5′HS4 boundary element blocks enhancer-mediated suppression of silencing. Molec. Cell. Biol. 19, 3714-37126.
38. Whitelaw, C. B., Springbett, A. J., Webster, J., Clark, A. J. (1993). The majority of GO transgenic mice are derived from mosaic embryos. Transgenic Res. 2, 29-32.
39. Wilkie, T. M., Brinster, R. L., Palmiter, R. D. (1986). Germline mosaicism and somatic in transgenic mice. Develop. Biol. 118:9-18. Williams, J. L., Ponce de Leon, F. A., Midura, P., Harrington, M., Meade, H., Echelard. Y. (1998). Analysis of multiple transgene integration sites in a beta casein antithrombin III goat line. Theriogenology 49, 398.
40. Williams, J. L., Pollock, D. P., lvlidura, P., Nims, S. D., Hawkins, N., Smith, T. E., Gavin, W. G., Meade, H. M., Echelard Y. (2000). FISH analysis of transgenic goats carrying two transgenes. Theriogenology 53:522.
41. Wilmut, I., Schnieke, A. E., McWhir, J., Kind, A. J., Campbell, K. H. S. (1999), Viable offspring derived from fetal and adult mammalian cells. Nature 385, 810-813.
42. Wright, G., Carver, A., Cottom, D., Reeves, D., Scott, A., Simons, P., Wilmut, I., Garner, I., Colman, A. (1991). High level expression of active human alpha-1-antitrypsin in the milk of transgenic sheep. Bio/Technology 9, 830-834.
43. Yong Z and L Yuqiang, (1998) Nuclear-Cytoplasmic Interaction and Development of Goat Embryos Reconstructed by Nuclear Transplantation: Production of Goats by Serially Cloning Embryos, BIOL REPROD.; 58: 266-269.
44. Zou X, et al., (2002) Generation Of Cloned Goats (Capra Hircus) From Transfected Foetal Fibroblast Cells, The Effect Of Donor Cell Cycle, MOL REPROD DEV.; 61: 164-172.

What is claimed is:

1. A method for the production of transgenic animals comprising:
transfecting a non-human mammalian pre-screened and characterized cell-line with a given transgene construct containing at least one DNA encoding a desired gene;
selecting a cell line(s) in which the desired gene has been inserted into the genome of that cell or cell-line;
performing a first nuclear transfer procedure to generate a first transgenic animal heterzygous for the desired gene; and,
characterizing the genetic composition of said first heterzygous transgenic animal.

2. The method of claim 1, wherein said first transgenic animal is biopsied so as to characterize the genome of said first transgenic animal.

3. The method of claim 2, wherein the cells or cell line biopsied from said first transgenic animal is expanded through cell culture techniques.

4. The method of claim 1, wherein said pre-screened cells are first characterized by one of several known molecular biology methods including without limitation FISH, Southern Blot, PCR.

5. The method of claim 1, wherein said donor differentiated mammalian cell to be used as a source of donor nuclei or donor cell nucleus is from an ungulate.

6. The method of claim 1, wherein the fetus develops into an offspring.

7. The method of claim 1, wherein said donor cell or donor cell nucleus is from an ungulate selected from the group consisting of bovine, ovine, porcine, equine, caprine and buffalo

8. The method of claim 5, wherein said donor cell or donor cell nucleus is from an ungulate selected from the group consisting of bovine, ovine, porcine, equine, caprine and buffalo.

9. The method of claim 1, wherein said donor differentiated mammalian cell to be used as a source of donor nuclei or donor cell nucleus is from an adult non-human mammalian somatic cell.

10. The method of claim 1, wherein said non-human mammal is a rodent.

11. The method of claim 1, wherein said donor differentiated mammalian cell to be used as a source of donor nuclei or donor cell nucleus is a non-quiescent somatic cell or a nucleus isolated from said non-quiescent somatic cell.

12. The method of claim 5, wherein the fetus develops into an offspring.

13. The resultant offspring of the methods of claim 1.

14. The resultant offspring of claim 1 further comprising wherein the offspring created as a result of said nuclear transfer procedure is homozygous for more than one desired gene.

15. The method of claim 1 further comprising using a second selective agent.

16. The method of claim 13 such that the transgenic pre-screened cell lines selected can proceed through a second or more multiple rounds selection to generate a cell line homozygous for more than one desired gene.

17. The method of claim 1, wherein cytocholasin-B is used in the cloning protocol.

18. The method of claim 1, wherein cytocholasin-B is not used in the cloning protocol.

19. The method of claim 1, wherein said donor differentiated mammalian cell to be used as a source of donor nuclei or donor cell nucleus is a non-quiescent somatic cell or a nucleus isolated from said non-quiescent somatic cell.

20. The resultant offspring of the method of claims 1.

21. The resultant offspring of the method of claim 17.

22. The resultant milk derived from the offspring of the method of claim 21.

23. The method of claim 1, wherein the desired gene codes for a biopharmaceutical protein product.

24. The method of claim 19 wherein said biopharmaceutical protein product is a compound selected from the group consisting of: Antithrombin III, lactoferrin, urokinase, PF4, alpha-fetoprotein, alpha-1-antitrypsin, C-1 esterase inhibitor, decorin, interferon, ferritin, transferring conjugates with biologically active peptides or fragments thereof, human serum albumin, prolactin, CFTR, blood Factor X, blood Factor VIII, as well as monoclonal antibodies.

25. The method of claim 1 wherein the DNA construct containing the desired gene is actuated by at least one beta casein promoter.

26. The resultant milk derived from the offspring of the method of claim 1.

27. The resultant milk derived from the offspring of the method of claim 21.
